(19) United States
(12) Patent Application Publication
(10) Pub. No.: US 2024/0122890 A1
(43) Pub. Date: Apr. 18, 2024

(54) USE OF EXOGENOUS KETONE ESTERS TO INDUCE WEIGHT LOSS IN MAMMALS

(71) Applicant: Buck Institute for Research on Aging, Novato, CA (US)

(72) Inventors: John C. Newman, San Francisco, CA (US); Brianna J. Stubbs, Richmond, CA (US)

(73) Assignee: Buck Institute for Research on Aging, Novato, CA (US)

(21) Appl. No.: 18/261,986

(22) PCT Filed: Jan. 24, 2022
(86) PCT No.: PCT/US2022/013533

§ 371 Date: Jul. 18, 2023

Related U.S. Application Data
(60) Provisional application No. 63/141,754, filed on Jan. 26, 2021.

(51) Int. Cl. A61K 31/225; A61K 31/121; A61K 47/12; A61K 47/46; A61P 3/04

(52) U.S. Cl. CPC A61K 31/225; A61K 31/121; A61K 47/12; A61K 47/46; A61P 3/04

(57) ABSTRACT

In various embodiments method and compositions are provided for inducing weight loss in a mammal. In certain embodiments the methods involve administering to a mammal an effective amount of a fatty acid β-hydroxyester compounds and/or one or more esters of butanediol.

### CROSS-REFERENCE TO RELATED APPLICATIONS

This application claims benefit of and priority to U.S. Ser. No. 63/141,754, filed on Jan. 26, 2021, which is incorporated herein by reference in its entirety for all purposes.

### STATEMENT OF GOVERNMENTAL SUPPORT [Not Applicable]

### BACKGROUND

Ketogenic diets and ketone bodies are of interest for the treatment of a variety of human disorders including epilepsy, dementia and diseases of aging. Ketone bodies are small compounds created from fat that serve as a substitute for sugar when the body's energy stores are depleted, such as when fasting or during strenuous exercise. Ketogenic diets stimulate the production of ketone bodies by containing very little sugar or other carbohydrates. The primary ketone bodies in humans are acetoacetate (AcAc) and β-hydroxybutyrate (BHB). Ketogenic diets are used clinically as a therapy for epilepsy, but they are often difficult to adhere to for long periods of time. The extremely high fat content (and low carbohydrate content) can make foods of a ketogenic diet unpalatable, and sometimes cause gastrointestinal problems, kidney stones, high cholesterol and other side effects.

BHB is a metabolic intermediate that is a currency for generating cellular energy, but also has several signaling functions separate from energy production. Either or both of the energy and signaling functions may be important for BHB's effects on human disease. During times of scarce glucose, for example during fasting or strenuous exercise, BHB is the currency by which energy stored in adipose tissue is turned into fuel that can be used by cells throughout the body to sustain their functions. Fat mobilized from adipose tissue is transported to the liver and converted into BHB. BHB circulates in the blood to all tissue. After being absorbed into a cell, BHB is broken down in the mitochondria to generate acetyl-CoA that is further metabolized into ATP. This is the canonical “energy currency” function of BHB.

In addition, BHB may have several signaling functions. Most of these are independent of its function as an energy currency, in that they are actions of the BHB molecule itself, and are not generally secondary effects of its metabolism into acetyl-CoA and ATP. Signaling functions may include: 1) inhibition of class I and IIa histone deacetylases, with resulting changes in histone modifications and gene expression, as well as changes in acetylation state and activity of non-histone proteins; 2) metabolism into acetyl-CoA results in increased cellular production of acetyl-coA to serve as substrate for acetyltransferase enzymes, resulting in similar changes in histone and non-histone protein acetylation as deacetylase inhibition; 3) covalent attachment to histones and possibly other proteins in the form of lysine-β-hydroxybutyrylation, which may have similar effects as lysine-acetylation; 4) binding and activation of hydroxycarboxylic acid receptor 2 (HCAR2) receptor with resultant alterations in adipose tissue metabolism; 5) binding and inhibition of free fatty acid receptor 3 (FFAR3) receptor with resultant changes in sympathetic nervous system activation and whole-body metabolic rate; and 6) inhibition of the NOD-like receptor 3 (NLRP3) inflammasome.

### SUMMARY

Various embodiments contemplated herein may include, but need not be limited to, one or more of the following:

Embodiment 1: A method of inducing weight loss in a mammal, said method comprising administering or causing to be administered to said mammal an effective amount of:
a compound according to Formula I:

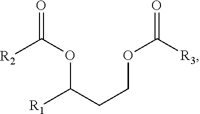

or salts, solvates, or hydrates thereof; and/or
a compound according to Formula II:

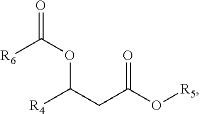

or salts, solvates or hydrates thereof, wherein:
   R₁ is H or C(1-6) alkyl or substituted alkyl;
   R₂ and R₃ are independently unsubstituted or substituted C(1-30) alkyl;
   R₄ is H or C(1-6) alkyl or substituted alkyl; and
   R₅ and R₆ are independently unsubstituted or substituted C(1-30) alkyl.

Embodiment 2: A method of altering the gut microbiome in a mammal, said method comprising administering or causing to be administered to said mammal an effective amount of:
a compound according to Formula I:

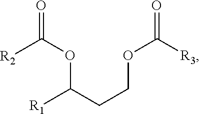

or salts, solvates, or hydrates thereof; and/or
a compound according to Formula II:

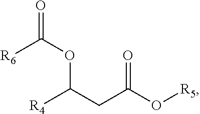

or salts, solvates or hydrates thereof, wherein:
   R₁ is H or C(1-6) alkyl or substituted alkyl;
   R₂ and R₃ are independently unsubstituted or substituted C(1-30) alkyl;
   R₄ is H or C(1-6) alkyl or substituted alkyl; and
   R₅ and R₆ are independently unsubstituted or substituted C(1-30) alkyl.

Embodiment 3: The method of embodiment 2, wherein said altering the gut microbiome comprises a reduction in intestinal Bifidobacterium.

Embodiment 4: A method of reducing blood glucose in a mammal, said method comprising administering or causing to be administered to said mammal an effective amount of:
a compound according to Formula I:

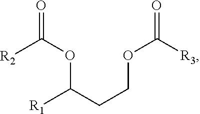

or salts, solvates, or hydrates thereof; and/or
a compound according to Formula II:

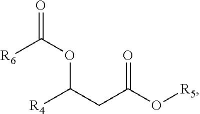

or salts, solvates or hydrates thereof, wherein:
   R₁ is H or C(1-6) alkyl or substituted alkyl;
   R₂ and R₃ are independently unsubstituted or substituted C(1-30) alkyl;
   R₄ is H or C(1-6) alkyl or substituted alkyl; and
   R₅ and R₆ are independently unsubstituted or substituted C(1-30) alkyl.

Embodiment 5: A method of reducing intestinal Th17 cell accumulation and/or inflammation in the intestine of a mammal, said method comprising administering or causing to be administered to said mammal an effective amount of:
a compound according to Formula I:

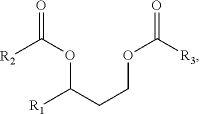

or salts, solvates, or hydrates thereof; and/or
a compound according to Formula II:

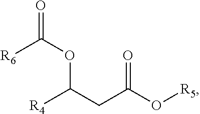

or salts, solvates or hydrates thereof, wherein:
   R₁ is H or C(1-6) alkyl or substituted alkyl;
   R₂ and R₃ are independently unsubstituted or substituted C(1-30) alkyl;
   R₄ is H or C(1-6) alkyl or substituted alkyl; and
   R₅ and R₆ are independently unsubstituted or substituted C(1-30) alkyl.

Embodiment 6: The method according to any one of embodiments 1-5, wherein said compound comprises a compound of Formula I:

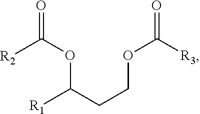

or salts, solvates or hydrates thereof.

Embodiment 7: The method of embodiment 6, wherein said compound is a compound of Formula Ia:

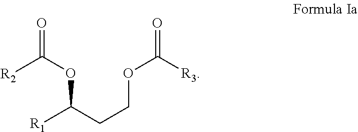

Embodiment 8: The method of embodiment 6, wherein said compound is a compound of Formula Ib:

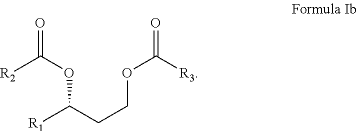

Embodiment 9: The method according to any one of embodiments 6-8, wherein R₁ is H.

Embodiment 10: The method according to any one of embodiments 6-8, wherein R₁ is C(1-6) alkyl or substituted alkyl.

Embodiment 11: The method of embodiment 10, wherein R₁ is C(1) alkyl or substituted alkyl.

Embodiment 12: The method of embodiment 10, wherein R₁ is C(2) alkyl or substituted alkyl.

Embodiment 13: The method of embodiment 10, wherein R₁ is C(3) alkyl or substituted alkyl.

Embodiment 14: The method of embodiment 10, wherein R₁ is C(4) alkyl or substituted alkyl.

Embodiment 15: The method of embodiment 10, wherein R₁ is C(5) alkyl or substituted alkyl.

Embodiment 16: The method of embodiment 10, wherein R₁ is C(6) alkyl or substituted alkyl.

Embodiment 17: The method according to any one of embodiments 6-8 and 10-16, wherein R₁ is unsubstituted alkyl.

Embodiment 18: The method according to any one of embodiments 6-8 and 10-17, wherein R₁ is a straight chain alkyl.

Embodiment 19: The method according to any one of embodiments 6-18, wherein R₂ is C(1-18) or C(1-6) alkyl or substituted alkyl.

Embodiment 20: The method of embodiment 19, wherein R₂ is C(1) alkyl or substituted alkyl.

Embodiment 21: The method of embodiment 19, wherein R₂ is C(2) alkyl or substituted alkyl.

Embodiment 22: The method of embodiment 19, wherein R₂ is C(3) alkyl or substituted alkyl.

Embodiment 23: The method of embodiment 19, wherein R₂ is C(4) alkyl or substituted alkyl.

Embodiment 24: The method of embodiment 19, wherein R₂ is C(5) alkyl or substituted alkyl.

Embodiment 25: The method of embodiment 19, wherein R₂ is C(6) alkyl or substituted alkyl.

Embodiment 26: The method of embodiment 19, wherein R₂ is C(7) alkyl or substituted alkyl.

Embodiment 27: The method of embodiment 19, wherein R₂ is C(8) alkyl or substituted alkyl.

Embodiment 28: The method according to any one of embodiments 6-27, wherein R₂ is an unsubstituted alkyl.

Embodiment 29: The method according to any one of embodiments 6-28, wherein R₂ is a straight chain alkyl.

Embodiment 30: The method according to any one of embodiments 6-29, wherein R₃ is C(1-18) or C(1-6) alkyl or substituted alkyl.

Embodiment 31: The method of embodiment 30, wherein R₃ is C(1) alkyl or substituted alkyl.

Embodiment 32: The method of embodiment 30, wherein R₃ is C(2) alkyl or substituted alkyl.

Embodiment 33: The method of embodiment 30, wherein R₃ is C(3) alkyl or substituted alkyl.

Embodiment 34: The method of embodiment 30, wherein R₃ is C(4) alkyl or substituted alkyl.

Embodiment 35: The method of embodiment 30, wherein R₃ is C(5) alkyl or substituted alkyl.

Embodiment 36: The method of embodiment 30, wherein R₃ is C(6) alkyl or substituted alkyl.

Embodiment 37: The method of embodiment 30, wherein R₃ is C(7) alkyl or substituted alkyl.

Embodiment 38: The method of embodiment 30, wherein R₃ is C(8) alkyl or substituted alkyl.

Embodiment 39: The method according to any one of embodiments 6-38, wherein R₃ is an unsubstituted alkyl.

Embodiment 40: The method according to any one of embodiments 6-39, wherein R₃ is a straight chain alkyl.

Embodiment 41: The method according to any one of embodiments 1-5, wherein said compound is selected from the group consisting of:

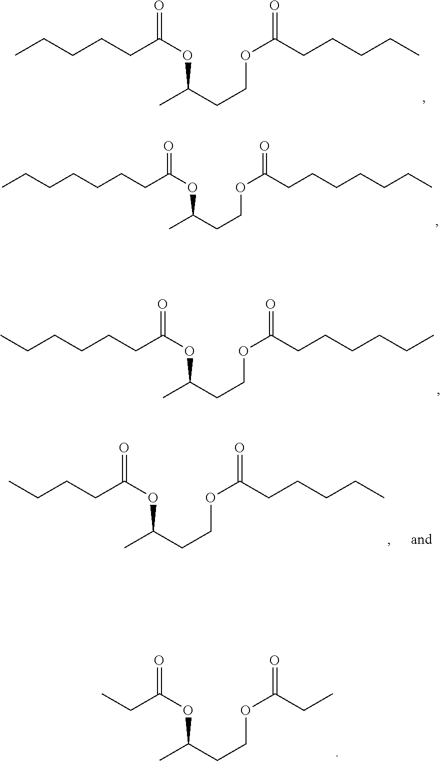

Embodiment 42: The method of embodiment 41, wherein said compound has the formula:

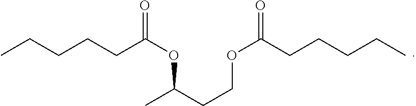 [SMILES: CCCCCC(=O)OCC[C@@H](C)OC(=O)CCCCC]

Embodiment 43: The method according to any one of embodiments 1-5, wherein said compound comprises a compound of Formula II:

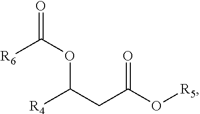

or salts, solvates or hydrates thereof.

Embodiment 44: The method of embodiment 43, wherein the compound is a compound of Formula IIa:

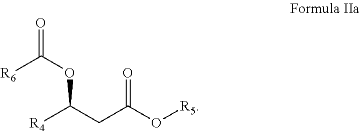

Embodiment 45: The method of embodiment 43, wherein the compound is a compound of Formula IIb:

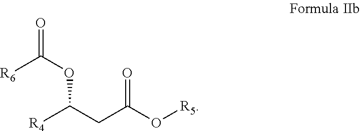

Embodiment 46: The method according to any one of embodiments 43-45, wherein R₄ is H.

Embodiment 47: The method according to any one of embodiments 43-45, wherein R₄ is C(1-6) alkyl or substituted alkyl.

Embodiment 48: The method of embodiment 47, wherein R₄ is C(1) alkyl or substituted alkyl.

Embodiment 49: The method of embodiment 47, wherein R₄ is C(2) alkyl or substituted alkyl.

Embodiment 50: The method of embodiment 47, wherein R₄ is C(3) alkyl or substituted alkyl.

Embodiment 51: The method of embodiment 47, wherein R₄ is C(4) alkyl or substituted alkyl.

Embodiment 52: The method of embodiment 47, wherein R₄ is C(5) alkyl or substituted alkyl.

Embodiment 53: The method of embodiment 47, wherein R₄ is C(6) alkyl or substituted alkyl.

Embodiment 54: The method according to any one of embodiments 43-45 and 47-53, wherein R₄ is unsubstituted alkyl.

Embodiment 55: The method according to any one of embodiments 43-45 and 47-54, wherein R₄ is straight chain alkyl.

Embodiment 56: The method according to any one of embodiments 43-55, wherein R₅ is C(1-18) or C(1-8) alkyl or substituted alkyl.

Embodiment 57: The method of embodiment 56, wherein R₅ is C(1) alkyl or substituted alkyl.

Embodiment 58: The method of embodiment 56, wherein R₅ is C(2) alkyl or substituted alkyl.

Embodiment 59: The method of embodiment 56, wherein R₅ is C(3) alkyl or substituted alkyl.

Embodiment 60: The method of embodiment 56, wherein R₅ is C(4) alkyl or substituted alkyl.

Embodiment 61: The method of embodiment 56, wherein R₅ is C(5) alkyl or substituted alkyl.

Embodiment 62: The method of embodiment 56, wherein R₅ is C(6) alkyl or substituted alkyl.

Embodiment 63: The method of embodiment 56, wherein R₅ is C(7) alkyl or substituted alkyl.

Embodiment 64: The method of embodiment 56, wherein R₅ is C(8) alkyl or substituted alkyl.

Embodiment 65: The method according to any one of embodiments 43-64, wherein R₅ is unsubstituted alkyl.

Embodiment 66: The method according to any one of embodiments 43-65, wherein R₅ is a straight chain alkyl.

Embodiment 67: The method according to any one of embodiments 43-66, wherein R₆ is C(1-18) or C(1-8) alkyl or substituted alkyl.

Embodiment 68: The method of embodiment 67, wherein R₆ is C(1) alkyl or substituted alkyl.

Embodiment 69: The method of embodiment 67, wherein R₆ is C(12 alkyl or substituted alkyl.

Embodiment 70: The method of embodiment 67, wherein R₆ is C(3) alkyl or substituted alkyl.

Embodiment 71: The method of embodiment 67, wherein R₆ is C(4) alkyl or substituted alkyl.

Embodiment 72: The method of embodiment 67, wherein R₆ is C(5) alkyl or substituted alkyl.

Embodiment 73: The method of embodiment 67, wherein R₆ is C(6) alkyl or substituted alkyl.

Embodiment 74: The method of embodiment 67, wherein R₆ is C(7) alkyl or substituted alkyl.

Embodiment 75: The method of embodiment 67, wherein R₆ is C(8) alkyl or substituted alkyl.

Embodiment 76: The method according to any one of embodiments 43-75, wherein R₆ is an unsubstituted alkyl.

Embodiment 77: The method according to any one of embodiments 43-76, wherein R₆ is a straight chain alkyl.

Embodiment 78: The method according to any one of embodiments 1-5, wherein said compound is selected from the group consisting of:

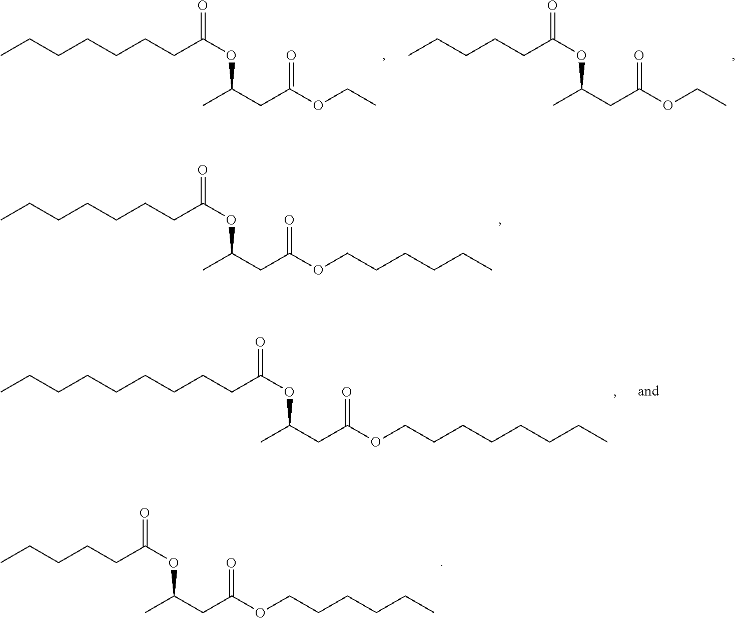

Embodiment 79: The method according to any one of embodiments 1-78, wherein said compound is administered as a component of a ketogenic diet.

Embodiment 80: The method of embodiment 79, wherein the ketogenic diet comprises a ratio by mass of fat to protein and carbohydrates of from about 2:1 to about 10:1.

Embodiment 81: The method of embodiment 80, wherein the ketogenic diet comprises a ratio by mass of fat to protein and carbohydrates of from about 3:1 to about 6:1.

Embodiment 82: The method according to any one of embodiments 1-78, wherein said compound is administered in a formulation wherein said formulation further comprises a ketone salt and/or a ketone free acid.

Embodiment 83: The method of embodiment 82, wherein said formulation comprises a ketone salt.

Embodiment 84: The method of embodiment 83, wherein said ketone salt is a ketone salt of said compound.

Embodiment 85: The method of embodiment 84, wherein said ketone salt is a ketone salt of bis hexanoyl (R)-1,3-butanediol.

Embodiment 86: The method according to any one of embodiments 83-85, wherein said formulation comprises said compound and said ketone salt in a ratio ranging from about 0.5:1 wt/wt (compound:ketone salt) to about 3:1 (compound:ketone salt), or from about 1:1 wt/wt (compound:ketone salt) to about 2:1 wt/wt (compound:ketone salt).

Embodiment 87: The method of embodiment 86, wherein said formulation comprises said compound and said ketone salt in a ratio of about 2:1 wt/wt (compound:ketone salt).

Embodiment 88: The method according to any one of embodiments 82-87, wherein said formulation comprises a ketone free acid.

Embodiment 89: The method of embodiment 88, wherein said formulation comprises a ketone free acid of said compound.

Embodiment 90: The method of embodiment 89, wherein said formulation comprises a ketone free acid of bis hexanoyl (R)-1,3-butanediol.

Embodiment 91: The method according to any one of embodiments 88-90, wherein said formulation comprises said compound and said ketone free acid in a ratio ranging from about 0.5:1 wt/wt (compound:ketone free acid) to about 3:1 (compound:ketone free acid), or from about 1:1 wt/wt (compound:ketone free acid) to about 2:1 wt/wt (compound:ketone free acid).

Embodiment 92: The method of embodiment 91, wherein said formulation comprises said compound and said ketone free acid in a ratio of about 2:1 wt/wt (compound:ketone free acid).

Embodiment 93: The method according to any one of embodiments 82-92, wherein said formulation further comprises citric acid.

Embodiment 94: The method according to any one of embodiments 82-93, wherein said formulation further comprises malic acid.

Embodiment 95: The method according to any one of embodiments 82-94, wherein said formulation further comprises a flavoring.

Embodiment 96: The method according to any one of embodiments 82-95, wherein said formulation further comprises a sweetener.

Embodiment 97: The method of embodiment 96, wherein said sweetener comprises monk fruit extract.

Embodiment 98: The method according to any one of embodiments 82-97, wherein said formulation further comprises a preservative.

Embodiment 99: The method of embodiment 98, wherein said formulation further comprises a preservative selected from the group consisting of potassium sorbate, and sodium benzoate.

### Definitions

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc.

A “therapeutically effective amount” or “efficacious amount” or “effective amount” refers to the amount of a compound that, when administered to a mammal or other subject is sufficient to provide the desired biological effect (e.g., weight loss). The “therapeutically effective amount” will vary depending on the compound and/or the age, weight, gender, etc., of the subject to be treated.

An “active agent” refers a chemical substance or compound that exerts a physiological action and is capable of treating, preventing or ameliorating one or more conditions/maladies and/or inducing one or more biological results (e.g., weight loss) as described herein. Examples of active agents of interest include fatty acid β-hydroxyester compounds (e.g., fatty acid esters of (β-hydroxybutyrate) and fatty acid esters of butanediol.

The nomenclature of certain compounds or substituents are used in their conventional sense, such as described in chemistry literature including but not limited to Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 30 carbon atoms (C(1-30). In certain illustrative, but non-limiting embodiments, an alkyl group comprises from 1 to 20 or from 1 to 10 carbon atoms. In certain embodiments, an alkyl group comprises from 1 to 8 carbon atoms, or from 1 to 6 carbon atoms, or from 1 to 4 carbon atoms.

The term “substituted” refers to a group in which one or more hydrogen atoms are independently replaced with the same or different substituent(s). Illustrative substituents include, but are not limited to, alkylenedioxy (such as methylenedioxy), —M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R₆₂ and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include —M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include —M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include —M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a C(1-4) alkyl group and a C(1-4) alkoxy group.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

The term “substantially pure” when used with respect to enantiomers indicates that one particular enantiomer (e.g., an S enantiomer) is substantially free of its stereoisomer. In various embodiments substantially pure indicates that a particular enantiomer is at least 70%, or at least 80%, or at least 90%, or at least 95%, or at least 98%, or at least 99% of the purified compound. Methods of producing substantially pure enantiomers are well known to those of skill in the art.

### BRIEF DESCRIPTION OF THE DRAWINGS

FIG. 1, illustrates the effect of added exogenous ketone esters (KE) on average body weight in mice. Food Change 1=Control food to mix of control and KE food. Food Change 2=Mix of control and KE food to 20% KE food alone

FIG. 2, panels A-B, illustrates the effect of added exogenous ketone esters (KE) on average body weight in male (panel A) and female (panel B) mice.

FIG. 3 illustrates waking body weight (lbs) over seven weeks with daily consumption of 25 g of ketone ester (KE).

### DETAILED DESCRIPTION

In various embodiments methods and compositions are provided for inducing weight loss in a mammal In certain embodiments weight loss is induced in a mammal by administering to the mammal one or more fatty acid β-hydroxyester compounds described herein and/or one or more esters of butanediol described herein. In some instances, the amount of the one or more of the fatty acid β-hydroxyester compounds and/or the one or more esters of butanediol administered to the subject is sufficient to induce weight loss in the mammal.

In various embodiments the one or more fatty acid β-hydroxyester compounds described herein and/or one or more esters of butanediol described herein can be administered to a mammal to alter the gut microbiome with concomitant effects on immune cells in the gut. Without being bound to a particular theory, it is believed that administration of the compounds described herein can modulate the microbiome in the gut (e.g., as illustrated by a reduction in intestinal Bifidobacterium) and can be associated concomitant downstream beneficial effects (e.g., a reduction in inflammation). In some instances, the amount of the one or more of the fatty acid β-hydroxyester compounds and/or the one or more esters of butanediol administered to the subject is sufficient to alter the microbiome and/or to reduce intestinal inflammation in a mammal.

In various embodiments the one or more fatty acid β-hydroxyester compounds described herein and/or one or more esters of butanediol described herein can be administered to a mammal to reduce intestinal Th17 cell accumulation and/or inflammation in the intestine of a mammal. In some instances, the amount of the one or more of the fatty acid β-hydroxyester compounds and/or the one or more esters of butanediol administered to the subject is sufficient reduce intestinal Th17 cell accumulation and/or inflammation in the intestine of a mammal

### Active Agent(s)—Ketone Ester(s)

In various embodiments the active agents used in the methods described herein comprise one or more compound(s) according to Formula I:

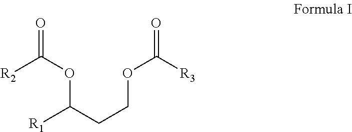

or salts, solvates, or hydrates thereof, where R₁ is H or C(1-8) or C(1-6) alkyl or substituted alkyl; and R₂ and R₃ are independently unsubstituted or substituted C(1-30), or C(1-12), or C(1-8), or C(1-6) alkyl; and/or one or more compound(s) according to Formula II

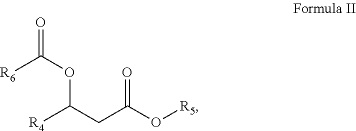

or salts, solvates or hydrates thereof, where R₄ is H or C(1-8) or C(1-6) unsubstituted alkyl or substituted alkyl; and R₅ and R₆ are independently unsubstituted or substituted C(1-30), or C(1-12), or C(1-8), or C(1-6) alkyl.

In certain embodiments the compound comprises a compound of Formula I. In certain embodiments the compound comprises a mixture of enantiomers. In certain embodiments the compound comprises predominantly (e.g., at least 80%, or at least 90%, or at least 95%, or at least 98%, by weight, a compound of Formula Ia:

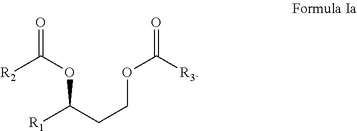

In certain embodiments the compound comprises predominantly (e.g., at least 80%, or at least 90%, or at least 95%, or at least 98%, by weight, a compound of Formula Ib:

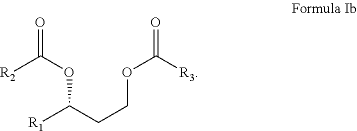

In certain embodiments R₁ is H. In certain embodiments R₁ is C(1-6) alkyl or substituted alkyl. In certain embodiments R₁ is C(1), or C(2), or C(3), or C(4), or C5), or C6 substituted alkyl or substituted alkyl. In certain embodiments R₁ is an unsubstituted alkyl. In certain embodiments R₁ is a straight chain alkyl. In certain embodiments R₂ is C(1), or C(2), or C(3), or C(4), or C(5), or C(6), or C(7), or C(8) unsubstituted alkyl or substituted alkyl. In certain embodiments R₂ is an unsubstituted alkyl. In certain embodiments R₂ is a straight chain alkyl. In certain embodiments R₃ is C(1), or C(2), or C(3), or C(4), or C(5), or C(6), or C(7), or C(8) unsubstituted alkyl or substituted alkyl. In certain embodiments R₃ is an unsubstituted alkyl. In certain embodiments R₃ is a straight chain alkyl.

In certain embodiments the compound is selected from the group consisting of:

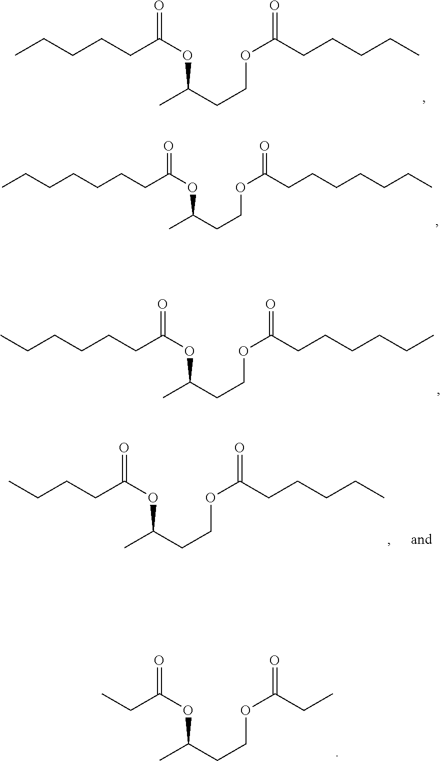

In certain embodiments the compound is bis hexanoyl (R)-1,3-butanediol (BH-BD):

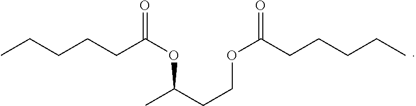 [SMILES: CCCCCC(=O)OCC[C@@H](C)OC(=O)CCCCC]

In certain embodiments the compound comprises a compound of Formula II. In certain embodiments the compound comprises a mixture of enantiomers. In certain embodiments the compound comprises predominantly (e.g., at least 80%, or at least 90%, or at least 95%, or at least 98%, by weight, a compound of Formula IIa:

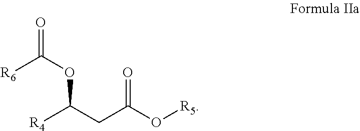

In certain embodiments the compound comprises predominantly (e.g., at least 80%, or at least 90%, or at least 95%, or at least 98%, by weight, a compound of Formula IIb:

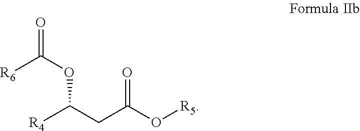

In certain embodiments R₄ is H. In certain embodiments R₄ is C(1-8), or C(1-6) alkyl or substituted alkyl. In certain embodiments R₄ is C(1), or C(2), or C(3), or C(4), or C(5), or C(6) unsubstituted alkyl or substituted alkyl. In certain embodiments R₄ is unsubstituted alkyl. In certain embodiments R₄ is a straight chain alkyl. In certain embodiments R₅ is C(1-18) or C(1-8) alkyl or substituted alkyl. In certain embodiments R₅ is C(1), or C(2), or C(3), or C(4), or C(5), or C(60, or C(7), or C(8) alkyl or substituted alkyl. In certain embodiments R₅ is an unsubstituted alkyl. In certain embodiments R₅ is a straight chain alkyl. In certain embodiments R₆ is C(1-18) or C(1-8) alkyl or substituted alkyl. In certain embodiments R₆ is C(1), or C(2), or C(3), or C(4), or C(5), or C(6), or C(7), or C(8) alkyl or substituted alkyl. In certain embodiments R₆ is an unsubstituted alkyl. In certain embodiments R₆ is a straight chain alkyl. In certain embodiments the compound comprises a compound selected from the group consisting of:

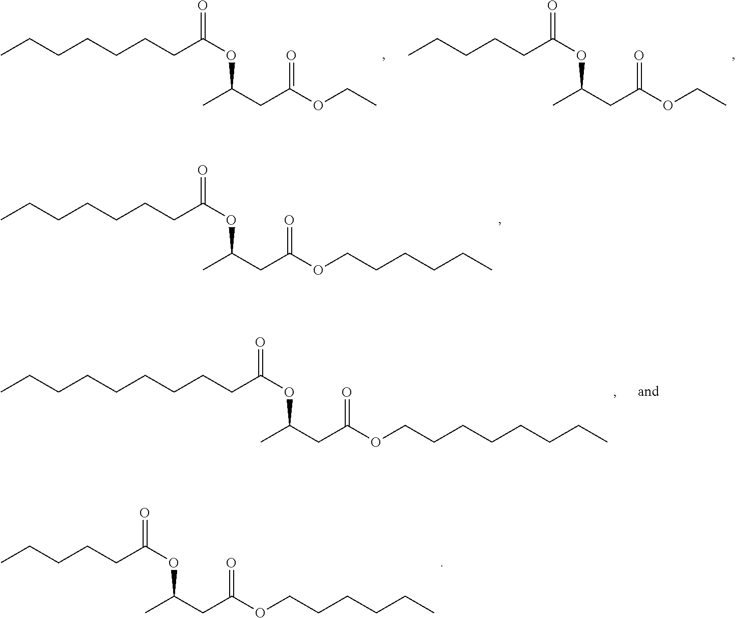

The compounds (e.g., ketone esters) described herein, may be prepared by chemical synthesis protocols known to those of skill in the art (See e.g., Green et al., “Protective Groups in Organic Chemistry,” (Wiley, 2nd ed. 1991); Harrison et al., “Compendium of Synthetic Organic Methods,” Vols. 1 8 (John Wiley and Sons, 1971 1996); “Beilstein Handbook of Organic Chemistry,” Beilstein Institute of Organic Chemistry, Frankfurt, Germany; Feiser et al., “Reagents for Organic Synthesis,” Volumes 1 17, (Wiley Interscience); Trost et al., “Comprehensive Organic Synthesis,” (Pergamon Press, 1991); “Theilheimer's Synthetic Methods of Organic Chemistry,” Volumes 1 45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well-established synthetic procedures.

In particular, detailed synthesis protocols for the compounds described herein are provided in PCT Patent Publication No: WO 2017/213999 A1 (PCT/US2017.035826) which is incorporated herein by reference for the synthesis protocols and compounds described therein. As taught therein, in illustrative, but non-limiting embodiments, β-hydroxyester compounds described herein may be obtained via synthetic routes as generally illustrated below:

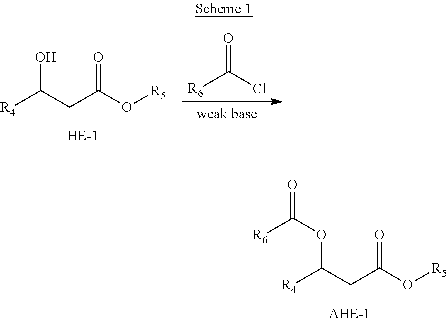

In Scheme 1, the hydroxyl group of β-hydroxyester HE-1 is deprotonated with a weak base (e.g., pyridine) and reacted with a substituted acyl chloride to give acyl-substituted β-hydroxyester AHE-1. R₄ may be H or a substituted or unsubstituted alkyl (e.g., C(1-8) alkyl, C(1-6) alkyl, etc.) and R₅ and R₆ are independently substituted or unsubstituted alkyl (e.g., C(1-30) alkyl, or C(20-20) alkyl).

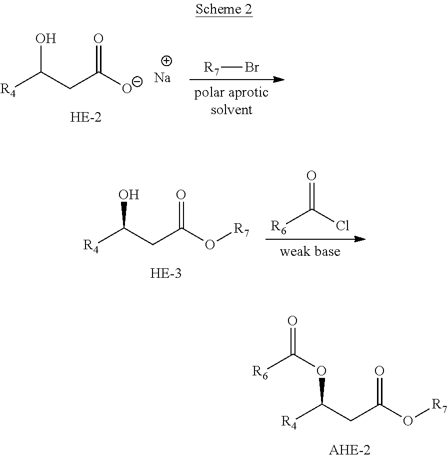

In Scheme 2, sodium β-hydroxyester HE-2 is reacted in a polar aprotic solvent (e.g., dimethylformamide) with an alkyl bromide to give alkyl β-hydroxyester HE-3. Deprotonation of the hydroxyl group of β-hydroxyester KE-3 with a weak base (e.g., pyridine) and reaction with a substituted acyl chloride gives acyl-substituted β-hyrdoxyester AHE-2. R₄ may be H or a substituted or unsubstituted alkyl (e.g., C(1-12), C(1-8), C(1-6) alkyl) and R₆ and R₇ are independently substituted or unsubstituted alkyl (e.g., C(1-30), or C(2-20) alkyl).

Also, as taught in Patent Publication No: WO 2017/213999 A1 1,3-butanediol esters described herein may be obtained via synthetic routes as generically illustrated below:

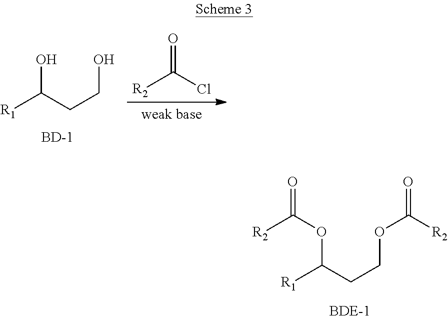

In Scheme 3, the hydroxyl groups of 1,3-butandiol BD-1 is deprotonated with a weak base (e.g., pyridine) and reacted with at least 2 equivalents of a substituted acyl chloride to give homo-acyl-substituted 1,3-butanediol ester BDE-1. R₁ may be H or a substituted or unsubstituted alkyl (e.g., C1-8), C(1-6), alkyl), and R₂ is substituted or unsubstituted alkyl (e.g., C(1-30), or C(2-20), or C(4-14)) alkyl.

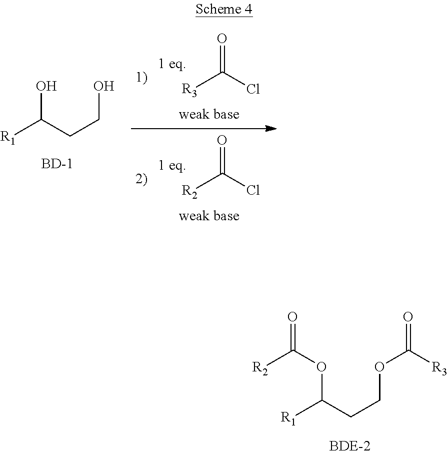

In Scheme 4, each hydroxyl group of 1,3-butandiol BD-1 is stepwise deprotonated with a weak base (e.g., pyridine) and reacted with 1 equivalent of a first substituted acyl chloride and 1 equivalent of a second substituted acyl chloride to give hetero-acyl-substituted 1,3-butanediol ester BDE-2. R₁ may be H or a substituted or unsubstituted alkyl (e.g., C(1-8) or C(1-6) alkyl), and R₂ and R₃ are independently substituted or unsubstituted alkyl (e.g., C(1-30), or C(2-20) alkyl).

These synthesis schemes are illustrative and non-limiting. Using the teaching provided herein and in PCT Publication No: WO 2017/213999 A1, numerous other synthesis protocols will be available to one of skill in the art.

### Components of a Ketogenic Diet

In various embodiments the compounds described herein are administered as components of a ketogenic diet. A ketogenic diet is a very low carbohydrate, high fat diet. Implementing a ketogenic diet involves significantly reducing carbohydrate intake and replacing it with fat. When this happens, the body becomes efficient at utilizing fat for energy production and turning fat into ketones in the liver that can supply energy for the brain.

Illustrative, but non-limiting ketogenic diets include, but are not limited to: 1) The standard ketogenic diet that is a low carbohydrate moderate protein and high fat diet typically containing about 70% fat, about 20% protein and only about 10% carbohydrates; and 2) The high protein ketogenic diet which is similar to a standard ketogenic diet, but includes more protein typically comprising a ratio of about 60% fat, about 35% protein, and about 5% carbohydrates. Exemplary ketogenic diets and components thereof are described for example in U.S. Pat. No. 6,207,856, the disclosure of which is incorporated by reference herein.

It will be recognized that the foregoing ketogenic diets are illustrative and non-limiting and using the teachings provided herein the compounds described herein can readily be used as components in a wide number of ketogenic diets.

### Pharmaceutical and/or Dietary Formulations

In certain embodiments the compounds described herein (e.g., bis hexanoyl (R)-1,3-butanediol (BH-BD)) may be formulated into a medicament or a dietary supplement by mixing with a dietetically or pharmaceutically acceptable carrier or excipient. In various embodiments, such a carrier or excipient may comprise, but is not limited to, a solvent, dispersion medium, coating, isotonic or absorption delaying agent, sweetener or the like. These include any and all solvents, dispersion media, coatings, isotonic and absorption delaying agents, sweeteners and the like. Suitable carriers may be prepared from a wide range of materials including, but not limited to, diluents, binders and adhesives, lubricants, disintegrants, coloring agents, bulking agents, flavoring agents, sweetening agents and miscellaneous materials such as buffers and adsorbents that may be needed in order to prepare a particular dosage form. The use of such media and agents for pharmaceutically active substances is well known in the art.

The compounds described herein (e.g., BH-BD) can be administered in the “native” form or, if desired, in the form of a derivative provided the derivative is suitable pharmacologically, e.g., effective in the present method(s). Derivatives of the compounds described herein can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of pharmaceutically formulating the compounds described herein as salts, esters, free acids, amides, and the like are well known to those of skill in the art. Certain salts can include, for example, halide salts. Certain basic salts include alkali metal salts, e.g., the sodium salt, and copper salts. Illustrative anionic salt forms include, but are not limited to acetate, benzoate, benzylate, bitartrate, bromide, carbonate, chloride, citrate, edetate, edisylate, estolate, formate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the compounds identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., amyloidogenic pathologies).

The active agent(s) described herein (e.g., BH-BD) can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the compound(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the compounds described herein, or excipients or other stabilizers and/or buffers.

Other physiologically acceptable materials, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g., calcium carbonate, carboxymethylcellulose calcium, sodium starch glycollate, crospovidone etc.), a binder (e.g., alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active agent(s) described herein (e.g., BH-BD) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) described herein and on the particular physio-chemical characteristics of the agent(s).

In certain embodiments, the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectable, implantable sustained-release formulations, mucoadherent films, topical varnishes, lipid complexes, etc.

Pharmaceutical compositions comprising the compounds described herein can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the compound(s) into preparations that can be used pharmaceutically. Proper formulation is dependent upon the route of administration chosen.

Systemic formulations include, but are not limited to, those designed for administration by injection, e.g., subcutaneous, intravenous, intramuscular, intrathecal or intraperitoneal injection, as well as those designed for transdermal, transmucosal oral or pulmonary administration. For injection, the compounds described herein can be formulated in aqueous solutions, preferably in physiologically compatible buffers such as Hanks solution, Ringer's solution, or physiological saline buffer and/or in certain emulsion formulations. The solution can contain formulatory agents such as suspending, stabilizing and/or dispersing agents. In certain embodiments, the compounds described herein can be provided in powder form for constitution with a suitable vehicle, e.g., sterile pyrogen-free water, before use. For transmucosal administration, penetrants appropriate to the barrier to be permeated can be used in the formulation. Such penetrants are generally known in the art.

For oral administration, the compounds can be readily formulated by combining the compound(s) with pharmaceutically acceptable carriers well known in the art. Such carriers enable the compounds described herein to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner

For administration by inhalation, the compounds described herein are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In various embodiments, the compounds described herein can be formulated in rectal or vaginal compositions such as suppositories or retention enemas, e.g., containing conventional suppository bases such as cocoa butter or other glycerides.

In addition to the formulations described previously, the compounds described herein may also be formulated as a depot preparation. Such long-acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to protect and deliver pharmaceutically active compounds. Certain organic solvents such as dimethylsulfoxide also can be employed, although usually at the cost of greater toxicity. Additionally, the compounds may be delivered using a sustained-release system, such as semipermeable matrices of solid polymers containing the therapeutic agent. Various uses of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

In certain embodiments, the compounds described herein (e.g., BH-BD) and/or formulations described herein are administered orally. This is readily accomplished by the use of tablets, caplets, lozenges, liquids, and the like.

In certain embodiments, the compound(s) and/or formulations described herein are administered systemically (e.g., orally, or as an injectable) in accordance with standard methods well known to those of skill in the art. In other embodiments, the agents can also be delivered through the skin using conventional transdermal drug delivery systems, e.g., transdermal “patches” wherein the compound(s) and/or formulations described herein are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the compounds and any other materials that are present.

In certain embodiments, one or more compounds described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In certain embodiments, the compounds described herein are suitable for oral administration. In various embodiments, the compound(s) in the oral compositions can be either coated or non-coated. The preparation of enteric-coated particles is disclosed for example in U.S. Pat. Nos. 4,786,505 and 4,853,230.

In various embodiments, compositions contemplated herein typically comprise one or more of the compound(s) described herein in an effective amount to achieve a pharmacological effect or therapeutic improvement (e.g., induction of weight loss) without undue adverse side effects. Illustrative pharmacological effects or therapeutic improvements include, but are not limited to, a reduction or cessation in the rate of bone resorption at one or more locations, an increase in bone density, a reduction in tumor volume, a reduction in arthritic pathology, and the like.

In various embodiments, the typical daily dose of compounds described herein (e.g., BH-BD) varies and will depend on various factors such as the individual requirements of the subjects to be treated. In certain embodiments, the daily dose of compounds can be in the range of 100-1,000 mg/kg, or from about 200-700 mg/kg, or from about 300-600 mg/kg. In one illustrative embodiment a standard approximate amount of the compounds described above present in the composition can be typically about 1 to about 10 g, or from 2 to about 5 g, or from about 2 to about 4 g. In certain embodiments the compounds are administered only once, or for follow-up as required. In certain embodiments the compounds and/or formulations thereof are administered once a day, in certain embodiments, administered twice a day, in certain embodiments, administered 3 times/day, and in certain embodiments, administered 4, or 6, or 6 or 7, or 8 times/day.

In certain embodiments the active agents described herein (e.g., BH-BD) are formulated in a single oral dosage form containing all active ingredients. Such oral formulations include solid and liquid forms. It is noted that solid formulations typically provide improved stability as compared to liquid formulations and can often afford better patient compliance.

In one illustrative embodiment, the one or more of the compounds described herein are formulated in a single solid dosage form such as single- or multi-layered tablets, suspension tablets, effervescent tablets, powder, pellets, granules or capsules comprising multiple beads as well as a capsule within a capsule or a double chambered capsule. In another embodiment, the compounds described herein (e.g., BH-BD) may be formulated in a single liquid dosage form such as suspension containing all active ingredients or dry suspension to be reconstituted prior to use.

In certain embodiments, the compounds described herein are formulated as enteric-coated delayed-release granules or as granules coated with non-enteric time-dependent release polymers in order to avoid contact with the gastric juice. Non-limiting examples of suitable pH-dependent enteric-coated polymers include, for example, cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. A suitable commercially available enteric material, for example, is sold under the trademark EUDRAGIT L 100-55®. This coating can be spray coated onto a substrate.

Illustrative non-enteric-coated time-dependent release polymers include, for example, one or more polymers that swell in the stomach via the absorption of water from the gastric fluid, thereby increasing the size of the particles to create thick coating layer. The time-dependent release coating generally possesses erosion and/or diffusion properties that are independent of the pH of the external aqueous medium. Thus, the active ingredient is slowly released from the particles by diffusion or following slow erosion of the particles in the stomach.

Illustrative non-enteric time-dependent release coatings are for example: film-forming compounds such as cellulosic derivatives, such as methylcellulose, hydroxypropyl methylcellulose (HPMC), hydroxyethylcellulose, and/or acrylic polymers including the non-enteric forms of the EUDRAGIT® brand polymers. Other film-forming materials can be used alone or in combination with each other or with the ones listed above. These other film forming materials generally include, for example, poly(vinylpyrrolidone), Zein, poly(ethylene glycol), poly(ethylene oxide), poly(vinyl alcohol), poly(vinyl acetate), and ethyl cellulose, as well as other pharmaceutically acceptable hydrophilic and hydrophobic film-forming materials. These film-forming materials may be applied to the substrate cores using water as the vehicle or, alternatively, a solvent system. Hydro-alcoholic systems may also be employed to serve as a vehicle for film formation.

Other materials suitable for making the time-dependent release coating of the compounds described herein include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include, but are not limited to poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

While the compounds and formulations thereof and methods of use thereof are described herein with respect to use in humans, they are also suitable for animal, e.g., veterinary use. Certain illustrative non-human organisms include, but are not limited to non-human primates, canines, equines, felines, porcines, rodents, ungulates, lagomorphs, and the like.

The foregoing formulations and administration methods are intended to be illustrative and not limiting. It will be appreciated that, using the teaching provided herein, other suitable formulations and modes of administration can be readily devised.

### Formulation Into Food Products and/or Dietary Supplements

When consumed, the compounds described herein (e.g., BH-BD) can be hydrolyzed into two products, hexanoic acid and 1,3-butanediol which can provide a calorie source that can be classified as a food and can form part of a food product.

A food product is an edible material composed primarily of one or more of the macronutrients protein, carbohydrate and fat, which is used in the body of an organism (e.g., a mammal) to sustain growth, repair damage, aid vital processes or furnish energy. A food product may also contain one or more micronutrients such as vitamins or minerals, or additional dietary ingredients such as flavorants and colorants.

Examples of food products into which the compounds described herein or compositions/formulations thereof may be incorporated as an additive include, but are not limited to snack bars, meal replacement bars, cereals, confectionery and probiotic formulations including, but not limited to yoghurts.

Examples of beverages and drinks include, but are not limited to, soft beverages, energy drinks, dry drink mixes, nutritional beverages, meal or food replacement drinks, compositions for rehydration (for instance during or after exercise), and teas (e.g., herbal teas) for infusion or herbal blends for decoction in water.

In certain embodiments a composition for rehydration typically comprises water, a sugar (or non-sugar sweetener), carbohydrate and one or more of the compounds described herein. In certain embodiments the composition may also comprise suitable flavorings, colorants and preservatives, as will be appreciated by one of skill in the art. The carbohydrate sugar, when present, can provide an energy source, and suitable sugars are known, including glucose and trehalose. In certain embodiments a meal or food replacement drink may be of the type commonly advocated for use in weight loss regimens. Such drink formulations typically comprise appropriate quantities of one or more macronutrients, i.e. sources of protein, fat and/or carbohydrate, together with optional additional ingredients such as solubilizing agents, preservatives, sweetening agents, flavoring agents and colorants.

A nutraceutical is a food ingredient, food supplement or food product that is considered to provide a medical or health benefit, including the prevention and treatment of disease. In general, a nutraceutical is specifically adapted to confer a particular health benefit on the consumer. In various embodiments a nutraceutical typically comprises a micronutrient such as a vitamin, mineral, herb, and/or phytochemical at a higher level than would be found in a corresponding regular (natural) food product. That level is typically selected to optimize the intended health benefit of the nutraceutical when taken either as a single serving or as part of a diet regimen or course of nutritional therapy. In certain embodiments the level would be a level effective to reduce plasma levels of fatty acids.

A functional food is a food that is marketed as providing a health benefit beyond that of supplying pure nutrition to the consumer. A functional food typically incorporates an ingredient such as a micronutrient as mentioned above, that confers a specific medical or physiological benefit other than a nutritional effect. A functional food typically carries a health claim on the packaging.

In certain embodiments a nutraceutical or functional food product typically contains the compounds described herein in an amount effective to lower plasma levels of free fatty acids in a subject. In certain embodiments a nutraceutical or functional food product typically contains the compounds described herein in an amount effective to lower blood glucose. More typically the nutraceutical or functional food product contains the compounds in an amount effective to suppress appetite, and/or to induce weight loss in a subject.

A dietary supplement is a product that is intended to supplement the normal diet of a subject (e.g., a human subject) and which contains a dietary ingredient such as a vitamin, mineral, herb or other botanical product, or amino acid. A dietary supplement is typically presented in unit dosage format and is designed for consumption with, before or after food but not in place of food. A dietary supplement is thus often presented as a tablet or capsule, or as dried powder or granules for sprinkling over food or adding to water or a beverage.

In certain embodiments one or more of the compounds according to Formula I and/or Formula II described herein are provided in a formulation where the formulation further comprises a ketone salt and/or a ketone free acid. In certain embodiments the formulation comprises a ketone salt. In certain embodiments the formulation comprises a ketone salt of the same compound(s) of Formula I or Formula II. In certain embodiments the ketone salt is a ketone salt of bis hexanoyl (R)-1,3-butanediol. In certain embodiments the formulation comprises the compound of Formula I and/or Formula II and the ketone salt in a ratio ranging from about 0.5:1 wt/wt (compound:ketone salt) to about 3:1 (compound:ketone salt), or from about 1:1 wt/wt (compound:ketone salt) to about 2:1 wt/wt (compound:ketone salt). In certain embodiments the formulation comprises the compound of Formula I and/or Formula II and the ketone salt in a ratio ranging of about 2:1 wt/wt (compound:ketone salt).

In certain embodiments the formulation comprises a ketone free acid. In certain embodiments the formulation comprises a ketone free acid of a compound of Formula I and/or Formula II. In certain embodiments the formulation comprises a ketone free acid of bis hexanoyl (R)-1,3-butanediol. In certain embodiments the formulation comprises the compound of Formula I and/or Formula II and the ketone free acid in a ratio ranging from about 0.5:1 wt/wt (compound:ketone free acid) to about 3:1 (compound:ketone free acid), or from about 1:1 wt/wt (compound:ketone free acid) to about 2:1 wt/wt (compound:ketone free acid). In certain embodiments the formulation comprises the compound of Formula I and/or Formula II and the ketone free acid in a ratio of about 2:1 wt/wt (compound:ketone free acid).

In certain embodiments the formulation further comprises citric acid and/or malic acid. In certain embodiments the formulation further comprises a flavoring (e.g., a natural flavoring such as raspberry flavoring). In certain embodiments the formulation further comprises a sweetener. In certain embodiments the sweetener comprises monk fruit extract. In certain embodiments the formulation comprises a preservative (e.g., potassium sorbate, sodium benzoate, etc.).

### EXAMPLES

The following examples are offered to illustrate, but not to limit the claimed invention.

### Example 1

### Ketone Esters Induce Weight Loss

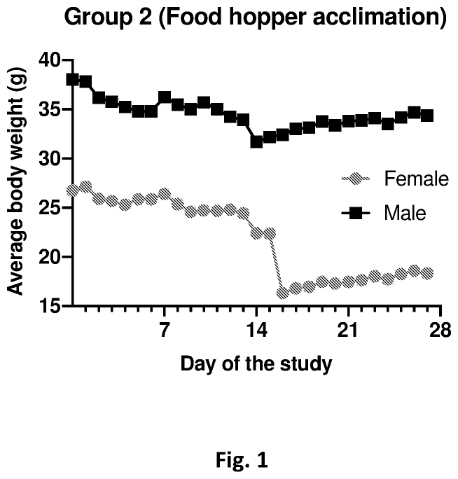

Eleven month old (middle aged) male (n=3) and female (n=3) C57/BL6 mice were fed control chow diet containing CHO 78.5%, PRO 10.5%, and FAT 12.5% for one week. The next week, mice were given a choice between control and 5% KE-food pellets (comprising bis hexanoyl (R)-1,3-butanediol (BH-BD))) for 3 days, followed by a choice between control and 20% KE-food pellets for 4 days. The control and KE food pellets were mixed together in the animal's food hopper. All mice were switched to a diet containing 20% KE (by weight) following this weeklong acclimation period. As shown in FIG. 1, males and females maintained a stable body weight during the acclimation period (presumably because they selectively ate the control diet pellets), and lost weight when switched to 20% KE-food on week 3.

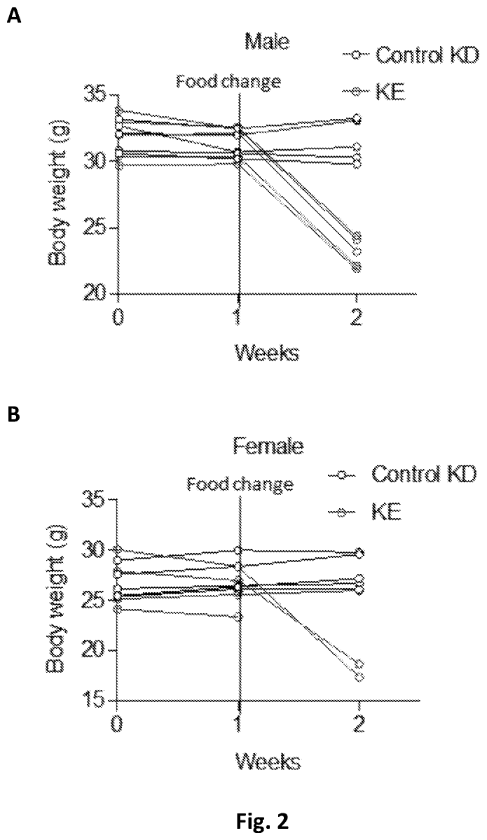

Male (n=10) and female (n=10) C57/BL6 mice were fed control diet for one week. The next week n=5 male and n=5 female mice were swapped to a diet containing 20% KE (by weight) mixed into a custom-made diet. All animals lost weight (see, FIG. 2, panels A and B). Two female animals died due to rapid weight loss.

### Example 2

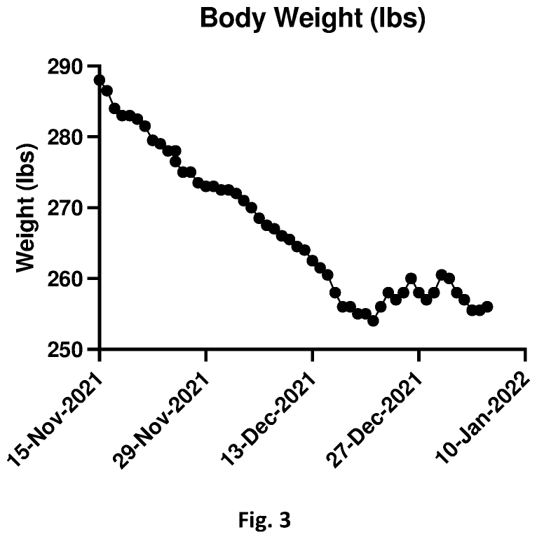

This example describes the effect of daily ketone ester consumption in a 58-year-old Caucasian male with obesity (BMI=42.5 kg/m²). The subject began consuming one 25 g serving of KE each morning on Nov. 15, 2021, and maintained daily consumption each morning for 7 weeks. The KE was in a commercially available consumer-friendly beverage formulation (Juvenescence, NJ, USA). Body weight was recorded by the subject after waking each morning (see, e.g., Table 1 and FIG. 3). The subject did not make any other deliberate diet or lifestyle changes, other than daily ketone ester consumption. The highest body weight in this period was on Nov. 15, 2021 (288 lbs), and the lowest was on Day Dec. 21, 2021 (254 lbs), giving a total weight loss of 34 lbs (11.8% of starting body mass)

TABLE 1
Raw data of waking body weight (lbs)
over 7 weeks with daily
consumption of 25 g of KE.

| Date | Weight (lbs) |
|---|---|
| 15 Nov. 2021 | 288 |
| 16 Nov. 2021 | 286.5 |
| 17 Nov. 2021 | 284 |
| 18 Nov. 2021 | 283 |
| 19 Nov. 2021 | 283 |
| 20 Nov. 2021 | 282.5 |
| 21 Nov. 2021 | 281.5 |
| 22 Nov. 2021 | 279.5 |
| 23 Nov. 2021 | 279 |
| 24 Nov. 2021 | 278 |
| 25 Nov. 2021 | 278 |
| 25 Nov. 2021 | 276.5 |
| 26 Nov. 2021 | 275 |
| 27 Nov. 2021 | 275 |
| 28 Nov. 2021 | 273.5 |
| 29 Nov. 2021 | 273 |
| 30 Nov. 2021 | 273 |
| 1 Dec. 2021 | 272.5 |
| 2 Dec. 2021 | 272.5 |
| 3 Dec. 2021 | 272 |
| 4 Dec. 2021 | 271 |
| 5 Dec. 2021 | 270 |
| 6 Dec. 2021 | 268.5 |
| 7 Dec. 2021 | 267.5 |
| 8 Dec. 2021 | 267 |
| 9 Dec. 2021 | 266 |
| 10 Dec. 2021 | 265.5 |
| 11 Dec. 2021 | 264.5 |
| 12 Dec. 2021 | 264 |
| 13 Dec. 2021 | 262.5 |
| 14 Dec. 2021 | 261.5 |
| 15 Dec. 2021 | 260.5 |
| 16 Dec. 2021 | 258 |
| 17 Dec. 2021 | 256 |
| 18 Dec. 2021 | 256 |
| 19 Dec. 2021 | 255 |
| 20 Dec. 2021 | 255 |
| 21 Dec. 2021 | 254 |
| 22 Dec. 2021 | 256 |
| 23 Dec. 2021 | 258 |
| 24 Dec. 2021 | 257 |
| 25 Dec. 2021 | 258 |
| 26 Dec. 2021 | 260 |
| 27 Dec. 2021 | 258 |
| 28 Dec. 2021 | 257 |
| 29 Dec. 2021 | 258 |
| 30 Dec. 2021 | 260.5 |
| 31 Dec. 2021 | 260 |
| 1 Jan. 2022 | 258 |
| 2 Jan. 2022 | 257 |
| 3 Jan. 2022 | 255.5 |
| 4 Jan. 2022 | 255.5 |
| 5 Jan. 2022 | 256 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inducing weight loss in a mammal, said method comprising administering or causing to be administered to said mammal an effective amount of:
a compound according to Formula I: 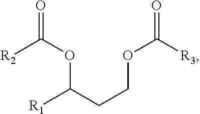
or salts, solvates, or hydrates thereof; and/or
a compound according to Formula II: 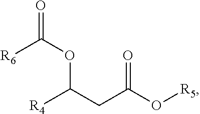
or salts, solvates or hydrates thereof, wherein:
R₁ is H or C(1-6) alkyl or substituted alkyl;
R₂ and R₃ are independently unsubstituted or substituted C(1-30) alkyl;
R₄ is H or C(1-6) alkyl or substituted alkyl; and
R₅ and R₆ are independently unsubstituted or substituted C(1-30) alkyl.

2. A method of altering the gut microbiome in a mammal, said method comprising administering or causing to be administered to said mammal an effective amount of:
a compound according to Formula I: 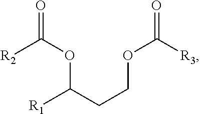
or salts, solvates, or hydrates thereof; and/or
a compound according to Formula II: 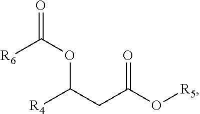
or salts, solvates or hydrates thereof, wherein:
R₁ is H or C(1-6) alkyl or substituted alkyl;
R₂ and R₃ are independently unsubstituted or substituted C(1-30) alkyl;
R₄ is H or C(1-6) alkyl or substituted alkyl; and
R₅ and R₆ are independently unsubstituted or substituted C(1-30) alkyl.

3. The method of claim 2, wherein said altering the gut microbiome comprises a reduction in intestinal Bifidobacterium.

4. A method of reducing blood glucose in a mammal, said method comprising administering or causing to be administered to said mammal an effective amount of:
a compound according to Formula I: 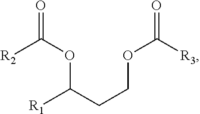
or salts, solvates, or hydrates thereof; and/or
a compound according to Formula II: 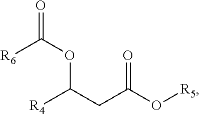
or salts, solvates or hydrates thereof, wherein:
R₁ is H or C(1-6) alkyl or substituted alkyl;
R₂ and R₃ are independently unsubstituted or substituted C(1-30) alkyl;
R₄ is H or C(1-6) alkyl or substituted alkyl; and
R₅ and R₆ are independently unsubstituted or substituted C(1-30) alkyl.

5. A method of reducing intestinal Th17 cell accumulation and/or inflammation in the intestine of a mammal, said method comprising administering or causing to be administered to said mammal an effective amount of:
a compound according to Formula I: 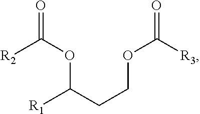
or salts, solvates, or hydrates thereof; and/or
a compound according to Formula II: 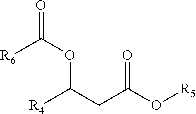
or salts, solvates or hydrates thereof, wherein:
R₁ is H or C(1-6) alkyl or substituted alkyl;
R₂ and R₃ are independently unsubstituted or substituted C(1-30) alkyl;
R₄ is H or C(1-6) alkyl or substituted alkyl; and
R₅ and R₆ are independently unsubstituted or substituted C(1-30) alkyl.

6. The method according to any one of claims 1-5, wherein said compound comprises a compound of Formula I: 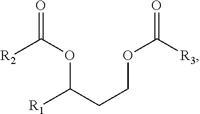
or salts, solvates or hydrates thereof.

7. The method of claim 6, wherein said compound is a compound of Formula Ia:

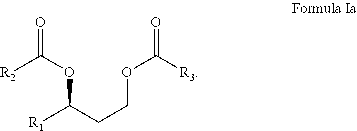

8. The method of claim 6, wherein said compound is a compound of Formula Ib:

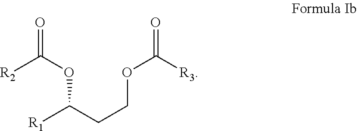

9. The method according to any one of claims 6-8, wherein R₁ is H.

10. The method according to any one of claims 6-8, wherein R₁ is C(1-6) alkyl or substituted alkyl.

11. The method of claim 10, wherein R₁ is C(1) alkyl or substituted alkyl.

12. The method of claim 10, wherein R₁ is C(2) alkyl or substituted alkyl.

13. The method of claim 10, wherein R₁ is C(3) alkyl or substituted alkyl.

14. The method of claim 10, wherein R₁ is C(4) alkyl or substituted alkyl.

15. The method of claim 10, wherein R₁ is C(5) alkyl or substituted alkyl.

16. The method of claim 10, wherein R₁ is C(6) alkyl or substituted alkyl.

17. The method according to any one of claims 6-8 and 10-16, wherein R₁ is unsubstituted alkyl.

18. The method according to any one of claims 6-8 and 10-17, wherein R₁ is a straight chain alkyl.

19. The method according to any one of claims 6-18, wherein R₂ is C(1-18) or C(1-6) alkyl or substituted alkyl.

20. The method of claim 19, wherein R₂ is C(1) alkyl or substituted alkyl.

21. The method of claim 19, wherein R₂ is C(2) alkyl or substituted alkyl.

22. The method of claim 19, wherein R₂ is C(3) alkyl or substituted alkyl.

23. The method of claim 19, wherein R₂ is C(4) alkyl or substituted alkyl.

24. The method of claim 19, wherein R₂ is C(5) alkyl or substituted alkyl.

25. The method of claim 19, wherein R₂ is C(6) alkyl or substituted alkyl.

26. The method of claim 19, wherein R₂ is C(7) alkyl or substituted alkyl.

27. The method of claim 19, wherein R₂ is C(8) alkyl or substituted alkyl.

28. The method according to any one of claims 6-27, wherein R₂ is an unsubstituted alkyl.

29. The method according to any one of claims 6-28, wherein R₂ is a straight chain alkyl.

30. The method according to any one of claims 6-29, wherein R₃ is C(1-18) or C(1-6) alkyl or substituted alkyl.

31. The method of claim 30, wherein R₃ is C(1) alkyl or substituted alkyl.

32. The method of claim 30, wherein R₃ is C(2) alkyl or substituted alkyl.

33. The method of claim 30, wherein R₃ is C(3) alkyl or substituted alkyl.

34. The method of claim 30, wherein R₃ is C(4) alkyl or substituted alkyl.

35. The method of claim 30, wherein R₃ is C(5) alkyl or substituted alkyl.

36. The method of claim 30, wherein R₃ is C(6) alkyl or substituted alkyl.

37. The method of claim 30, wherein R₃ is C(7) alkyl or substituted alkyl.

38. The method of claim 30, wherein R₃ is C(8) alkyl or substituted alkyl.

39. The method according to any one of claims 6-38, wherein R₃ is an unsubstituted alkyl.

40. The method according to any one of claims 6-39, wherein R₃ is a straight chain alkyl.

41. The method according to any one of claims 1-5, wherein said compound is selected from the group consisting of:

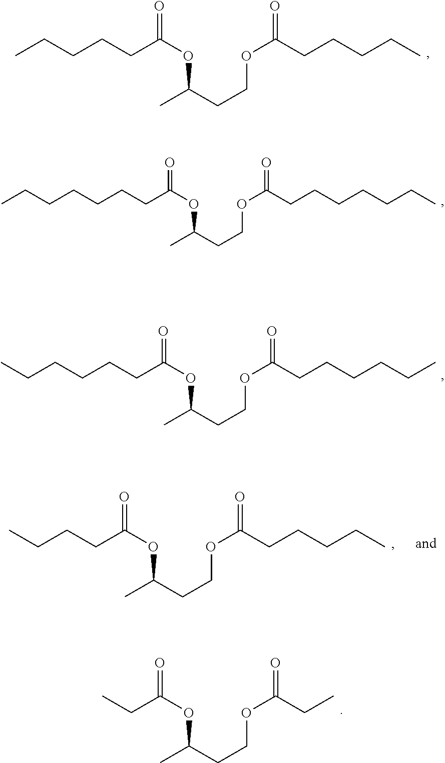

42. The method of claim 41, wherein said compound has the formula:

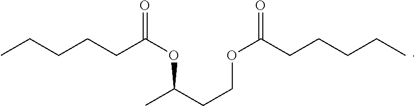 [SMILES: CCCCCC(=O)OCC[C@@H](C)OC(=O)CCCCC]

43. The method according to any one of claims 1-5, wherein said compound comprises a compound of Formula II: 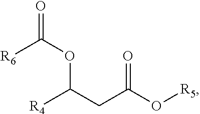
or salts, solvates or hydrates thereof.

44. The method of claim 43, wherein the compound is a compound of Formula IIa:

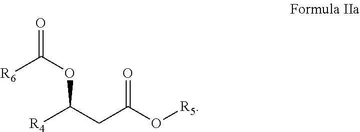

45. The method of claim 43, wherein the compound is a compound of Formula IIb:

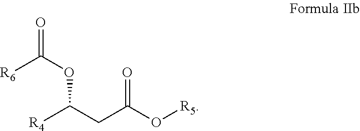

46. The method according to any one of claims 43-45, wherein R₄ is H.

47. The method according to any one of claims 43-45, wherein R₄ is C(1-6) alkyl or substituted alkyl.

48. The method of claim 47, wherein R₄ is C(1) alkyl or substituted alkyl.

49. The method of claim 47, wherein R₄ is C(2) alkyl or substituted alkyl.

50. The method of claim 47, wherein R₄ is C(3) alkyl or substituted alkyl.

51. The method of claim 47, wherein R₄ is C(4) alkyl or substituted alkyl.

52. The method of claim 47, wherein R₄ is C(5) alkyl or substituted alkyl.

53. The method of claim 47, wherein R₄ is C(6) alkyl or substituted alkyl.

54. The method according to any one of claims 43-45 and 47-53, wherein R₄ is unsubstituted alkyl.

55. The method according to any one of claims 43-45 and 47-54, wherein R₄ is straight chain alkyl.

56. The method according to any one of claims 43-55, wherein R₅ is C(1-18) or C(1-8) alkyl or substituted alkyl.

57. The method of claim 56, wherein R₅ is C(1) alkyl or substituted alkyl.

58. The method of claim 56, wherein R₅ is C(2) alkyl or substituted alkyl.

59. The method of claim 56, wherein R₅ is C(3) alkyl or substituted alkyl.

60. The method of claim 56, wherein R₅ is C(4) alkyl or substituted alkyl.

61. The method of claim 56, wherein R₅ is C(5) alkyl or substituted alkyl.

62. The method of claim 56, wherein R₅ is C(6) alkyl or substituted alkyl.

63. The method of claim 56, wherein R₅ is C(7) alkyl or substituted alkyl.

64. The method of claim 56, wherein R₅ is C(8) alkyl or substituted alkyl.

65. The method according to any one of claims 43-64, wherein R₅ is unsubstituted alkyl.

66. The method according to any one of claims 43-65, wherein R₅ is a straight chain alkyl.

67. The method according to any one of claims 43-66, wherein R₆ is C(1-18) or C(1-8) alkyl or substituted alkyl.

68. The method of claim 67, wherein R₆ is C(1) alkyl or substituted alkyl.

69. The method of claim 67, wherein R₆ is C(12 alkyl or substituted alkyl.

70. The method of claim 67, wherein R₆ is C(3) alkyl or substituted alkyl.

71. The method of claim 67, wherein R₆ is C(4) alkyl or substituted alkyl.

72. The method of claim 67, wherein R₆ is C(5) alkyl or substituted alkyl.

73. The method of claim 67, wherein R₆ is C(6) alkyl or substituted alkyl.

74. The method of claim 67, wherein R₆ is C(7) alkyl or substituted alkyl.

75. The method of claim 67, wherein R₆ is C(8) alkyl or substituted alkyl.

76. The method according to any one of claims 43-75, wherein R₆ is an unsubstituted alkyl.

77. The method according to any one of claims 43-76, wherein R₆ is a straight chain alkyl.

78. The method according to any one of claims 1-5, wherein said compound is selected from the group consisting of:

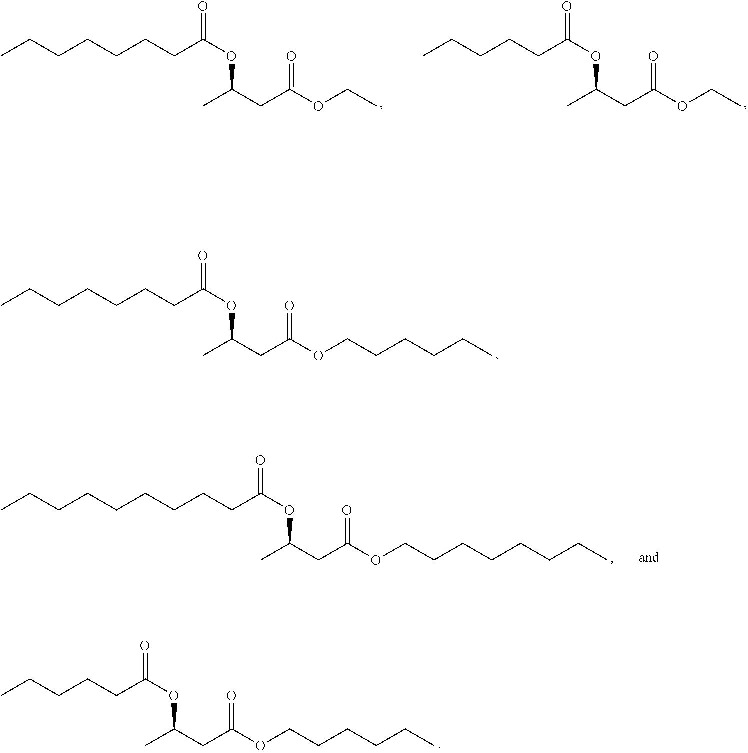

79. The method according to any one of claims 1-78, wherein said compound is administered as a component of a ketogenic diet.

80. The method of claim 79, wherein the ketogenic diet comprises a ratio by mass of fat to protein and carbohydrates of from about 2:1 to about 10:1.

81. The method of claim 80, wherein the ketogenic diet comprises a ratio by mass of fat to protein and carbohydrates of from about 3:1 to about 6:1.

82. The method according to any one of claims 1-78, wherein said compound is administered in a formulation wherein said formulation further comprises a ketone salt and/or a ketone free acid.

83. The method of claim 82, wherein said formulation comprises a ketone salt.

84. The method of claim 83, wherein said ketone salt is a ketone salt of said compound.

85. The method of claim 84, wherein said ketone salt is a ketone salt of bis hexanoyl (R)-1,3-butanediol.

86. The method according to any one of claims 83-85, wherein said formulation comprises said compound and said ketone salt in a ratio ranging from about 0.5:1 wt/wt (compound:ketone salt) to about 3:1 (compound:ketone salt), or from about 1:1 wt/wt (compound:ketone salt) to about 2:1 wt/wt (compound:ketone salt).

87. The method of claim 86, wherein said formulation comprises said compound and said ketone salt in a ratio of about 2:1 wt/wt (compound:ketone salt).

88. The method according to any one of claims 82-87, wherein said formulation comprises a ketone free acid.

89. The method of claim 88, wherein said formulation comprises a ketone free acid of said compound.

90. The method of claim 89, wherein said formulation comprises a ketone free acid of bis hexanoyl (R)-1,3-butanediol.

91. The method according to any one of claims 88-90, wherein said formulation comprises said compound and said ketone free acid in a ratio ranging from about 0.5:1 wt/wt (compound:ketone free acid) to about 3:1 (compound:ketone free acid), or from about 1:1 wt/wt (compound:ketone free acid) to about 2:1 wt/wt (compound:ketone free acid).

92. The method of claim 91, wherein said formulation comprises said compound and said ketone free acid in a ratio of about 2:1 wt/wt (compound:ketone free acid).

93. The method according to any one of claims 82-92, wherein said formulation further comprises citric acid.

94. The method according to any one of claims 82-93, wherein said formulation further comprises malic acid.

95. The method according to any one of claims 82-94, wherein said formulation further comprises a flavoring.

96. The method according to any one of claims 82-95, wherein said formulation further comprises a sweetener.

97. The method of claim 96, wherein said sweetener comprises monk fruit extract.

98. The method according to any one of claims 82-97, wherein said formulation further comprises a preservative.

99. The method of claim 98, wherein said formulation further comprises a preservative selected from the group consisting of potassium sorbate, and sodium benzoate.
